SEQUENCE LISTING (10 sequences)

SEQ ID NO: 1 - DNA, 33 bp, Artificial Sequence
  Description of Artificial Sequence Synthetic oligonucleotide
tttttagcatatgactatgttactcgctatagc

SEQ ID NO: 2 - DNA, 33 bp, Artificial Sequence
  Description of Artificial Sequence Synthetic oligonucleotide
gtacttgcaatatgtgcaatggcgaggattttt

SEQ ID NO: 3 - DNA, 60 bp, Artificial Sequence
  Description of Artificial Sequence Synthetic oligonucleotide
aatcctcgccattgcacatattgcaagtacgctatagcgagtaacatagtcatatgctaa

SEQ ID NO: 4 - DNA, 60 bp, Artificial Sequence
  Description of Artificial Sequence Synthetic oligonucleotide
aatcgtatactgatacaatgagcgatatcgcatgaacgttatacacgttaccgctcctaa

SEQ ID NO: 5 - DNA, 10 bp, Artificial Sequence
  Description of Artificial Sequence Synthetic oligonucleotide
cggcatgcat

SEQ ID NO: 6 - DNA, 10 bp, Artificial Sequence
  Description of Artificial Sequence Synthetic oligonucleotide
atgcatgccg

SEQ ID NO: 7 - DNA, 10 bp, Artificial Sequence
  Description of Artificial Sequence Synthetic oligonucleotide
gtgcagactt

SEQ ID NO: 8 - DNA, 10 bp, Artificial Sequence
  Description of Artificial Sequence Synthetic oligonucleotide
aagtctgcac

SEQ ID NO: 9 - DNA, 20 bp, Artificial Sequence
  Description of Artificial Sequence Synthetic oligonucleotide
gtgcagaccttgtgaacgcc

SEQ ID NO: 10 - DNA, 20 bp, Artificial Sequence
  Description of Artificial Sequence Synthetic oligonucleotide
ggcgttcacaaggtctgcac

(19) United States
(12) Patent Application Publication
(10) Pub. No.: US 2014/0024818 A1
(43) Pub. Date: Jan. 23, 2014

(54) ALIGNMENT OF NANOMATERIALS AND MICROMATERIALS

(71) Applicants: Yi Lu, Champaign, IL (US); Jung Heon Lee, Champaign, IL (US); Mehmet Veysel Yigit, Urbana, IL (US)

(72) Inventors: Yi Lu, Champaign, IL (US); Jung Heon Lee, Champaign, IL (US); Mehmet Veysel Yigit, Urbana, IL (US)

(21) Appl. No.: 11/939,226

(22) Filed: Nov. 13, 2007

Related U.S. Application Data
(60) Provisional application No. 60/865,744, filed on Nov. 14, 2006.

(51) Int. Cl. C07H 21/00

(52) U.S. Cl. CPC C07H 21/00; Y10S 977/773; Y10S 977/882; B82Y 40/00
USPC 536/23.1; 977/773; 977/882

(57) ABSTRACT

The present invention provides a method for preparing a nanoassembly that includes the step of reacting the assembly template with at least one nanomaterial to form the nanoassembly using a bifunctional linker.

### REFERENCE TO RELATED APPLICATIONS

This application claims the benefit of U.S. Provisional Application No. 60/865,744 entitled “Alignment of Nanomaterials and Micromaterials” filed Nov. 14, 2006, which is incorporated by reference in its entirety.

### FEDERALLY SPONSORED RESEARCH OR DEVELOPMENT

This subject matter of this application may have been funded in part under the following research grants and contracts: National Science Foundation Grant Nos. DMI-0328162 and DMR-0117792, and United States Department of Defense Grant No. DAAD19-03-1-0227. The U.S. Government may have rights in this invention.

### BACKGROUND

Recent progress in materials science has led to the development of singly functional nanomaterials such as nanoparticles. The ordered assembly of multifunctional nanomaterials is central to the development of integrated circuits designed for nanoelectronics, photonics, magnetics, such as spintronics, biosensors, and programmable or autonomous molecular machines. Furthermore, such functional nanomaterials are envisioned for use in integrated circuits adapted for nanoscale sensor arrays (Hagleitner et al. 2001), field-programmable gate-arrays (Heath et al. 1998), and cellular nonlinear networks (Yang et al. 2001). At the present time, however, the implementation of multifunctional nanomaterials in these application areas is limited owing to the lack of predictable assembly methods for these materials.

To enable the application of nanomaterials suitable to these areas, assembly methodologies for generating multifunctional nanomaterials are required. Furthermore, the assembly of multifunctional nanoparticles into hierarchical structures having unique spatial resolution and functional specificities will become necessary for the aforementioned applications. In particular, multifunctional nanomaterials with controlled spatial resolution and high specificity will be important to permit template-directed assembly using “one-pot” procedures.

Nucleic acid polymers represent attractive candidate templates upon which multifunctional nanomaterials may be assembled. Nucleic acid polymers form predictable two-dimensional secondary structures based upon the complementary base-pairing relationships established between the purine and pyrimidine nucleobases. Furthermore, nucleic acid polymers can form three-dimensional tertiary structures of predictable specificity, shape and form that rely upon the hydrogen-bonding interactions between the nucleobases as well as base-stacking interactions between individual base-pairs. Provided that the derivatization and subsequent functionalization of the nucleic acid polymer do not interfere with its ability to form secondary and tertiary structures, a nucleic acid polymer represents a suitable candidate template for the assembly of multifunctional nanomaterials.

In general, however, nucleic acid polymers have not been extensively used as templates for nanomaterial development because no systematic approach existed whereby nanomaterials could be precisely aligned along the polymer. Owing to the redundant nature of the monomeric subunits that comprise a typical nucleic acid polymer, only the 5′ and 3′ termini represent unique structures of any given nucleic acid molecule. The internal phosphodiester bonds that link the individual nucleotides within a nucleic acid polymer are identical in chemical composition and are not readily amenable to modification in a site-specific manner. Furthermore, the nucleobases offer limited functional groups that are amenable to chemical modification, as most functional groups of nucleobases participate in hydrogen-bonding interactions which are responsible for the secondary and tertiary structures formed. While nucleic acid polymers can form predictable two-dimensional and three-dimensional structures, the paucity of available unique sites within nucleic acids has rendered them less than practical templates for the development of multifunctional nanomaterials.

### SUMMARY

In a first aspect, the invention is a method for preparing a nanoassembly that includes the step of reacting an assembly template with at least one nanomaterial to form the nanoassembly.

In a second aspect, the invention is a nanoassembly that includes an assembly template and a nanomaterial.

In a third aspect, the invention is a multifunctional nanoassembly that includes an assembly template, a first nanomaterial, and a second nanomaterial.

In the fourth aspect, the invention is a method for preparing a multifunctional nanoassembly having a first nanomaterial and a second nanomaterial, which includes the steps of reacting the first nanomaterial with an assembly template and of reacting the second nanomaterial with the assembly template.

In a fifth aspect, the invention is a method for preparing a microassembly that includes reacting an assembly template with a micromaterial to form the microassembly.

In a sixth aspect, the invention is an assembly that includes a polymer template and a material, where the material comprises at least one member selected from the group consisting of a nanomaterial and a micromaterial.

In a seventh aspect, the invention is a multifunctional assembly that includes a polymer template, a first material, and a second material. The first and second materials include at least one member selected from the group consisting of a nanomaterial and a micromaterial.

In an eighth aspect, the invention is a method for preparing a multifunctional assembly having a first material and a second material that includes reacting the first material with an assembly template and reacting the second material with the assembly template.

### DEFINITIONS

The term “particle” includes nanoparticle and microparticle.

The term “aspect ratio” means the ratio of the longest axis of an object to the shortest axis of the object, where the axes are not necessarily perpendicular.

The term “longest axis” of a particle means the longest straight distance between two points on the surface of the particle. For example, a helical particle would have a longest axis corresponding to the length of the particle in its helical conformation.

The term “longest dimension” of a particle means the longest direct path of the particle. The term “direct path” means the shortest path contained within the particle between two points on the surface of the particle. For example, a helical would have a longest dimension corresponding to the length of the helix if it were stretched out into a straight line.

The term “width” of a cross-section is the longest dimension of the cross-section, and the “height” of a cross-section is the dimension perpendicular to the width.

The “width” of a particle means the average of the widths of the particle; and the “diameter” of a particle means the average of the diameters of the particle.

The “average” dimension of a plurality of particles means the average of that dimension for the plurality. For example, the “average diameter” of a plurality of nanospheres means the average of the diameters of the nanospheres, where a diameter of a single nanosphere is the average of the diameters of that nanosphere.

The term “nanoparticle” means a particle with at least two dimensions of 100 nanometers (nm) or less.

The term “nanosphere” means a nanomaterial having an aspect ratio of at most 3:1.

The term “nanorod” means a nanomaterial having a longest dimension of at most 200 nm, and having an aspect ratio of from 3:1 to 20:1.

The term “nanotube” means a nanomaterial having a hollow interior and a diameter between 0.1 and 100 nm and having an aspect ratio of greater than 3:1.

The term “nanofiber” means a nanomaterial having a longest dimension greater than 200 nm, and having an aspect ratio greater than 20:1.

The term “nanowire” means a nanofiber having a longest dimension greater than 1 μm.

The term “nanobelt” means a nanofiber having a cross-section in which the ratio of the width to the height of the cross-section is at least 2:1.

The term “nanosheet” means a nanobelt in which the ratio of the width of the cross-section to the height of the cross-section is at least 20:1.

The term “nanocard” means a nanoparticle having a cross-section in which the ratio of the width of the cross-section to the height of the cross-section is at least 2:1, and having a longest dimension less than 100 nm.

The term “nanoprism” means a nanoparticle having at least two non-parallel faces connected by a common edge.

The term “nanonetwork” means a plurality of individual nanomaterials that are interconnected.

The phrase “quantum dot” refers to a semiconductor crystal that contains 100 to 100,000 atoms and ranges from 2 to 10 nanometers in diameter. Examples of semiconductors include CdSe, ZnS, and CeTe. Additional examples of quantum dots are described in U.S. Patent Publication No. U.S. Pat. No. 6,939,604 B1, entitled DOPED SEMICONDUCTOR NANOCRYSTALS, to Guyot-Sionnest et al.

The term “microparticle” means a particle with at least two dimensions of greater than 100 nm, preferably in the range between 100 nm and 100 micrometers (μm).

The term “microsphere” means a micromaterial having an aspect ratio of at most 3:1.

The term “microrod” means a micromaterial having a longest dimension greater than 200 nm, and having an aspect ratio of from 3:1 to 20:1.

The term “microtube” means a micromaterial having hollow interior and a diameter greater than 100 nm and having an aspect ratio of greater than 3:1.

The term “microfiber” refers to a fiber that is one denier or less and has a diameter 100 nm or more and an aspect ratio greater than 20:1.

The term “microwire” means a microfiber having a longest dimension greater than 1 μm.

The term “microbelt” means a microfiber having a cross-section in which the ratio of the width to the height of the cross-section is at least 2:1.

The term “microsheet” means a microbelt in which the ratio of the width of the cross-section to the height of the cross-section is at least 20:1.

The term “microcard” means a micromaterial having a cross-section in which the ratio of the width of the cross-section to the height of the cross-section is at least 2:1, and having a longest dimension 100 nm or more.

The term “microprism” means a micromaterial having at least two non-parallel faces connected by a common edge.

The term “micronetwork” means a plurality of individual micromaterials that are interconnected.

The term “nanomaterials” includes nanoparticles; nanospheres; nanorods; nanotubes; nanofibers, including nanowires, nanobelts, and nanosheets; nanocards; and nanoprisms; and these nanoparticles may be part of a nanonetwork. The term “nanomaterial” refers to a collection of a particular type of nanoparticle, quantum dot, etc. For example, a collection of gold nanoparticles, gold nanospheres, gold nanofibers, or gold nanorods would each be a gold nanomaterial.

The term “micromaterials” includes microparticles; microspheres; microrods; microtubes; microfibers, including microwires, microbelts, and microsheets; microcards; and microprisms; and these microparticles may be part of a micronetwork. The term “micromaterial” refers to a collection of a particular type of microparticle. For example, a collection of gold microparticles, gold microspheres, gold microfibers, or gold microrods would each be a gold micromaterial.

The term “assembly” includes nanoassembly and microassembly.

The term “nanoassembly” refers to an assembly template that is coupled to at least one nanomaterial.

The term “microassembly” refers to an assembly template that is coupled to at least one micromaterial.

The phrase “multifunctional assembly” includes multifunctional nanoassembly and multifunctional microassembly.

The phrase “multifunctional nanoassembly” refers to an assembly template that is coupled to at least two different types of nanomaterials. In the context of describing methods and materials common to their synthesis, the term “nanoassembly” includes “multifunctional nanoassembly.” For example, methods suitable for the synthesis of a multifunctional nanoassembly, as described herein, will also be suitable for the synthesis of a nanoassembly.

The phrase “multifunctional microassembly” refers to an assembly template that is coupled to at least two different types of micromaterials. In the context of describing methods and materials common to their synthesis, the term “microassembly” includes “multifunctional microassembly.” For example, methods suitable for the synthesis of a multifunctional microassembly, as described herein, will also be suitable for the synthesis of a microassembly. As used herein, methods suitable for the synthesis of a multifunctional nanoassembly and a nanoassembly, as described herein, will also be suitable for the synthesis of a multifunctional microassembly and a microassembly.

The phrase “assembly template” refers to a chemically-modified polymer to which one or more nanomaterials or micromaterials may be chemically coupled. An assembly template may be composed of a single-stranded molecule or double-stranded molecule. An assembly template includes products from a reaction between a polymer containing at least one reactive substituent and at least one linking reagent. The reaction between a polymer and a linking reagent occurs between at least one reactive substituent of the polymer and a reactive group of the linking reagent.

The phrase “linking reagent” refers to a molecule having a first reactive group and a second reactive group separated by a linker segment. In the context of the present invention, following reaction between A (for example, a polymer) and B (for example, a nanomaterial) with a linking reagent, the product includes A and B that are coupled together through a moiety containing the linker segment.

The phrase “linking agent” is a moiety containing one reactive group. The product of the reaction between A and a linking reagent contains A coupled to a linking agent. In the context of the present invention, following reaction between A (for example, a polymer) with a linking reagent, the product (for example, an assembly template) includes A coupled to a linker agent having a linker segment and a reactive group.

The terms “DNA,” “RNA,” and “PNA” refer to deoxyribonucleic acid, ribonucleic acid, and peptide nucleic acid, respectively.

The phrase “nucleic acid polymer” or “polymer” refers to a natural or synthetic chemical molecule having at least three nucleosides covalently-coupled together through chemical linkage of their ribose moieties. Examples of the chemical linkages between nucleosides include phosphodiester, phosphorothioate, phosphoselenoate, and phosphoroamide, among others. Examples of a nucleic acid polymer include DNA, RNA, and derivatives thereof, including mixed systems (for example, and in any order of organization, DNA-RNA co-polymers, PNA-RNA co-polymers, PNA-DNA co-polymers, and PNA-RNA-DNA co-polymers).

The phrase “peptide nucleic acid” comprises a polyamide backbone (for example, N-(2-aminoethyl)glycine) and nucleoside bases (available from, for example, Biosearch, Inc. (Bedford, Mass.)).

The phrase “polymer template” refers to a moiety containing a polymer chain having nucleobase side groups.

The phrase “reactive substituent” refers to a natural or synthetic nucleobase or backbone component of a nucleic acid polymer that is chemically altered to include another moiety that is reactive. Reactive substituents are typically positioned at an internal site within a polymer for subsequent reaction with a suitable linking reagent. Where reactive substituents are positioned at the 5′ and/or 3′ termini of a polymer, at least one reactive substituent will exist additionally within the polymer.

The phrase “coupling activity preference” refers to the specificity of a reactive group of a linker agent for coupling to a first material relative to a second material. For example, a thiol group may display greater specificity for coupling to a gold nanoparticle relative to a silver nanoparticle.

The phrase “surface fixing reagent,” refers to a molecule that can react with a surface to form a chemical attachment between the surface and the molecule.

### BRIEF DESCRIPTION OF THE DRAWINGS

FIG. 1 depicts the two-part reaction scheme between a linking reagent, a polymer containing a phosphorothioate group, and a gold nanoparticle;

FIG. 2 (a) depicts UV-vis spectra of Au nanoparticles functionalized with phosphorothioate modified DNA strand by a linking reagent following hybridization to a complementary DNA strand (dashed line tracing), or following denaturation of the resultant hybrid (solid line tracing), or following incubation in the presence of a non-complementary DNA strand (dotted line tracing, overlapping the solid line tracing);

FIG. 2(b) depicts UV-vis spectra of a mixture containing Au nanoparticles and a DNA strand lacking a phosphorothioate modified site following incubation in the presence of a complementary DNA strand (dashed line tracing) or a non-complementary DNA strand (solid line tracing). The non phosphorothioate modified DNA was treated with linking reagents in the same way;

FIG. 2(c) depicts UV-vis spectra of a mixture containing Au nanoparticles and a DNA strand containing a phosphorothioate modified site following incubation in the presence of a complementary DNA strand (dashed line tracing) or a non-complementary DNA strand (solid line tracing) without a linking reagent;

FIG. 3(a) depicts TOF-MS scan of a DNA polymer containing a reactive substituent (phosphorothioate);

FIG. 3(b) depicts TOF-MS scan of an assembly template composed of a DNA polymer containing a reactive substituent (phosphorothioate) and a linker reagent; and

FIG. 4 depicts scanning electron microscopy images of gold nanoparticles assembled onto phosphorothioate-modified DNA polymers.

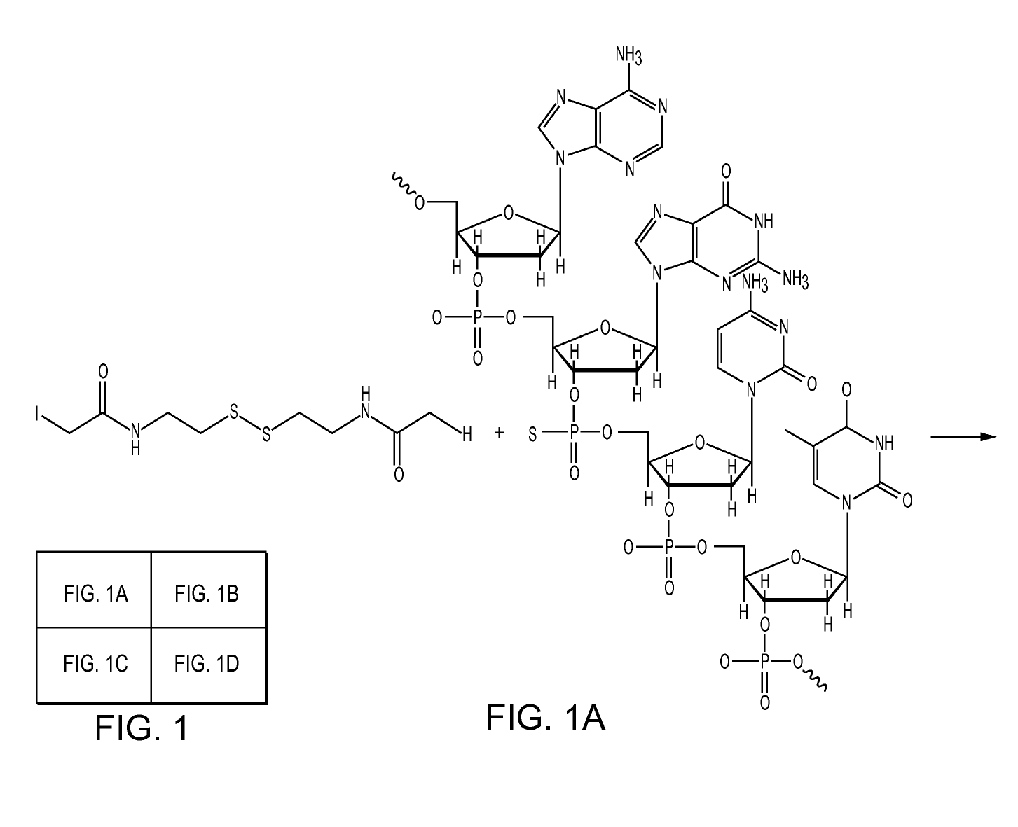

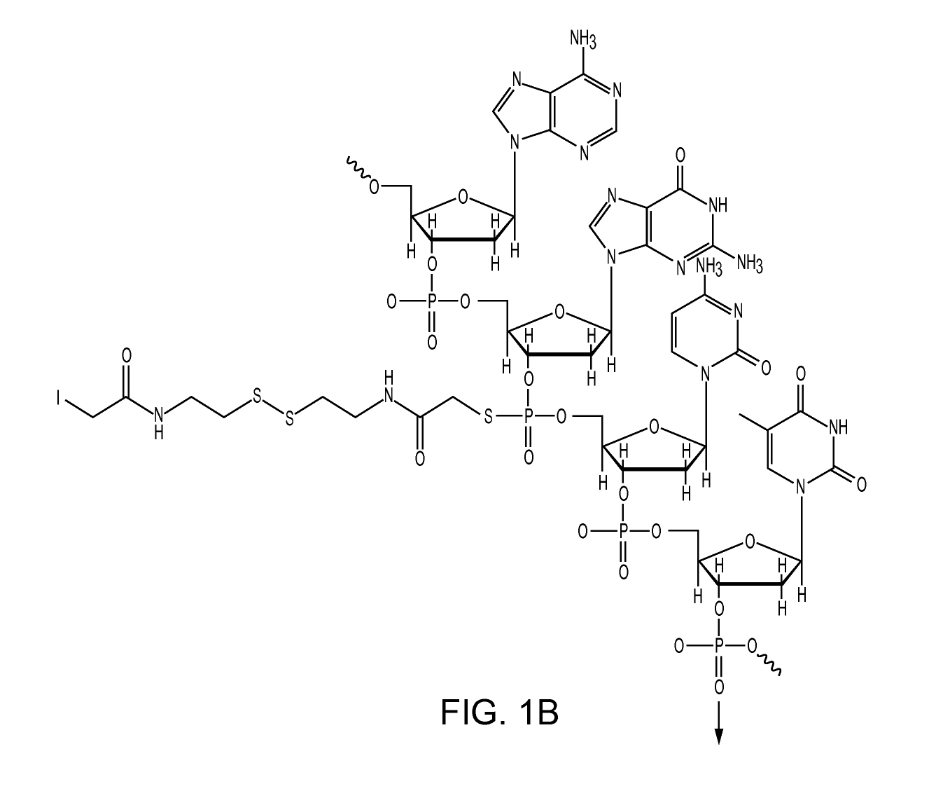

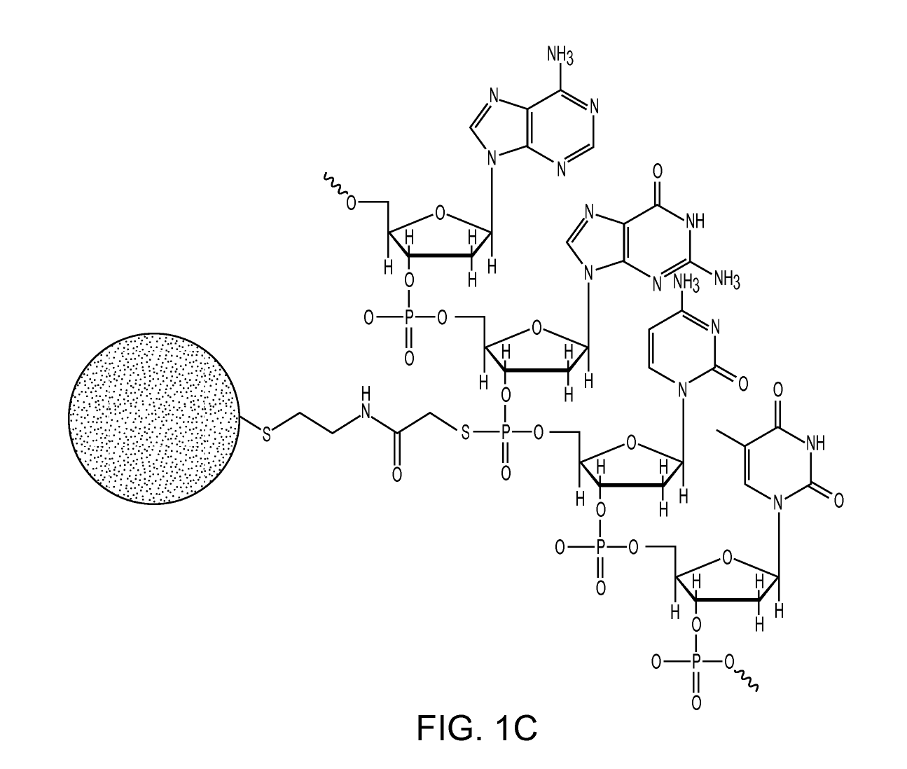

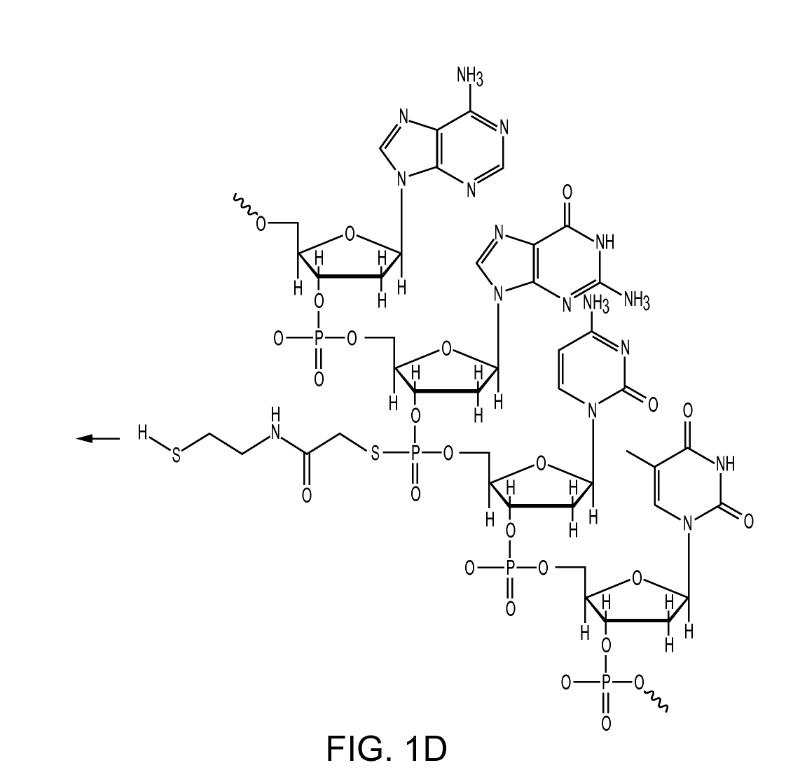

### DETAILED DESCRIPTION

The present invention makes use of the discovery of practical methods that permit precise coupling of a nanomaterial on a modified phosphodiester linkage at a defined position in a nucleic acid polymer. In particular, the present invention makes use of the finding that nucleic acid polymers can be prepared using standard synthetic chemical methods that incorporate at precise positions one or more reactive substituents into the phosphodiester backbone of the nucleic acid polymer that can be modified subsequently to incorporate a nanomaterial. Nucleic acid polymers serve as novel design and assembly templates for the present invention owing to their ability to form definite and predictable two-dimensional (secondary) and three-dimensional (tertiary) structures. Thus, the present invention provides methods for assembling a defined number of multifunctional nanomaterials at defined positions in polymers that can form two-dimensional arrays and three-dimensional structures.

The present invention circumvents problems associated with the conventional use of the phosphodiester linkage as a site for the precise alignment of nanomaterials along a nucleic acid polymer. The methods described provide the ability to uniquely position, in a sequence-defined manner, a precise number of nanoparticles on the phosphodiester backbone of a nucleic acid polymer. By virtue of being able to predict the two-dimensional and three-dimensional configuration of the resultant nucleic acid polymer, the complete structural configuration of the nanoassemblies may be defined. Furthermore, methods are described that provide for multifunctional nanoassemblies using nucleic acid polymers as nanomaterial design templates that represent clear advances over the previous approaches to nanomaterial design and assembly.

Multifunctional nanoassemblies may be manufactured using chemically-tailored nucleic acid polymers as assembly templates. Nucleic acid polymers are chemically synthesized so as to include at least one protected reactive substituent located at one or more precise positions along the polymer. The modified nucleic acid polymer may then be deprotected, purified, and subsequently reacted with a linking reagent at the sites carrying the reactive substituent to generate the assembly template bearing at least one linking agent at one or more precise positions. Nanoassemblies are then prepared by reacting the assembly template with one or more nanomaterials.

The nucleic acid polymers of the present invention are assembled using routine synthetic chemical procedures. A reactive substituent is introduced into the site chosen for incorporation of the nanomaterial during the synthesis process. For example, the use of a phosphoramidite modified to contain a phosphorothioate permits site-specific incorporation of phosphorothioate into the nucleic acid polymer during the synthesis of the polymer. Preferred modified phosphoramidites include phosphoramidites containing phosphorothioate, phosphoselenoate, or phosphoroamide. Because the inclusion of the modified phosphoroamidite is defined according to the implemented synthetic program for the desired nucleic acid polymer, the position of the modification sites in the nucleic acid polymer is precisely determined.

Following deprotection and purification of the nucleic acid polymer, the next step of the assembly process is reaction of the modified site in the polymer with a suitable linking reagent. The ultimate purpose of the linking reagent is to couple a nanomaterial via a linker segment to a modified site in the nucleic acid polymer that contains the reactive substituent. The choice of the linking reagent will depend upon the chemical nature of the modified site in the nucleic acid polymer as well as the composition of the nanomaterial. For example, where the design objective is to couple a gold (Au) nanoparticle to a nucleic acid polymer containing a phosphorothioate modification, an appropriate linking reagent may contain a first reactive group that permits its chemical linkage to the reactive sulfur substituent of the phosphorothioate (for example, a iodoacetamide group) and a second reactive group that permits its chemical linkage with Au of the nanoparticle (for example, a thiol group). Preferred linking reagents include Dithio-bis-succinimidyl propionate (Lomant's reagent), N-succinimidyl-(4-iodoacetyl)aminobenzoate, 3-maleimidopropionic acid (NHS), sulfo-SIAB, N-succinimidyl S-acetylthioacetate, N-succinimidyl S-acetylthiopropionate succinimidyl iodoacetate, succinimidyl bromoacetate and Succinimidyl-6-(iodoacetyl)aminocaproate, which are available from commercial sources (e.g., Pierce, Rockford, Ill., Molecular Biosciences, Inc., Boulder, Colo., etc.).

Additional preferred linking reagents that bind to phosphorothioate groups include bromo- [symbol] -unsaturated carbonyls, iodo (or bromo) acetamides, aziridinyl sulfonamides, and molecules such as 3-(2-iodoacetamido)-2,2,5,5-tetramethyl-1-pyrrolidinyloxy (PROXYL), monobromobimane, 4-bromocrotonic acid, [symbol] -bromo- [symbol] -unsaturated carbonyl dihydropyrroloindole, bromoacetamido dihydropyrroloindole, and N-dansylaziridine, among others.

Linking reagents suitable to one of the preferred embodiments include the same reactive groups at their termini and thiols protected in an internal disulfide bond as second reactive groups. An especially preferred linking reagent for reaction with phosphorothioate modified DNA is N,N′-Bis (□-isoacetyl)-2,2′-dithiobis(ethylamine) (BIDBE). Such linking reagents are useful for nucleic acid polymers bearing more than one reactive substituent.

Following reaction of this linking reagent with a nucleic acid polymer containing, for example, reactive substituents at two sites, many possible reaction products are possible in a single-pot reaction, including: (1) a lariat polymer structure where the two sites within the polymer are linked together through a linker segment; (2) a linear polymer structure that contains two linking agents singly-coupled at each site within the polymer; (3) a multimeric polymer structure that contains two or more polymers crosslinked together through one or more linker segments; and potentially other structured products. The ratio of these species attained from the reaction can be controlled in part by adjusting the linking reagent:polymer ratio of the reaction. For example, linear polymers substituted with linking agents can be favored by providing an excess of linking reagent relative to polymer. However, such reaction conditions result only in enrichment of the desired linear polymer products at the expense of the linking reagent as a wasted reactant.

The choice of a linking reagent such as BIDBE in the reaction simplifies resolution of complex products and improves reaction yield of the desired assembly templates. Inclusion of the disulfide bond in the linking reagent enables any additional structures besides the desired linear polymer product to be resolved as the desired linear polymer product. Following completion of the reaction that chemically couples the linking reagent to the polymer, the reaction mixture is treated under conditions to reduce the internal disulfide bond to form free thiols. The deprotected thiols are then available for reaction with nanomaterials.

Suitable reducing reagents for this reaction include thiourea, dithiothreitol, glutathione, tris(2-carboxyethyl)phosphine hydrochloride (TCEP), among others. A preferred reducing reagent is tris(2-carboxyethyl)phosphine hydrochloride.

Protection of the second reactive group in linkers like BIDBE also improves reaction efficiency and economy for generating assembly templates. For example, the iodoacetamide group not only has reactivity for a phosphorothioate group of a polymer, but it also displays reactivity for the alkane thiol group of the linking reagent. Thus, linkers containing the thiol groups protected in the form of disulfides preclude them from eliminating the linking reagents as reactants due to self-reaction between the first and second reactive groups. The alkane thiol groups need not be protected in linkers containing as a first reactive group an amine or carboxyl group, as these groups are not prone to reaction with the free alkane thiols. Any linking reagent that contains a thiol group as a second reactive group can be synthesized as a disulfide to protect this group from participating in unwanted reactions during conjugation of the linking reagent to the polymer.

Preferred nanoassemblies should remain stable for their intended applications. Assembly templates can display condition-dependent, linker stability profiles. For example, a polymer that has a BIDBE linker agent coupled to a phosphorothioate-modified site displays a marked pH-dependent stability profile. Mass spectrometry studies of treated and untreated polymer-BIDBE conjugates reveal that the linker agent remains stably coupled to the polymer at pH 5 but is decoupled from the polymer at pH 7. Thus, studies of the stability profile of polymer-linker agent conjugates under a variety of conditions relevant to the synthesis and the intended application of the nanoassembly should be conducted to maximize the yield and stability of the desired products.

Preferred linking agents do not interfere with the ability of nucleic acid polymers to form regular secondary and tertiary structures. In this regard, the linking reagent should enable attachment to a reactive substituent in the nucleic acid polymer in a manner that avoids chemical or steric interference with the nucleobases of the polymer. Furthermore, the resultant linking agent should provide the ability to couple to a nanomaterial without distorting or disrupting the secondary structure or tertiary structure adopted by the underlying nucleic acid polymer. The linking agent should have adequate clearance from the proximity of the phosphodiester backbone to enable efficient chemical coupling to nanomaterials. Thus, preferred linking reagents will have a linker segment separating the first reactive group and the second reactive group by a distance ranging from 2 Å to 50 Å. A preferred linker segment includes linear and branched alkyl groups, saturated and unsaturated alkyl groups, amides, amines, ethers, esters, and the like.

The nucleic acid polymers of the present invention may be synthesized using automated procedures commonly available and known in the art. An example of a commonly used nucleic acid synthesizer suitable for the present invention includes the Applied Biosystems 381A Automated DNA Synthesizer. Custom-synthesized polymers may be obtained from a variety of commercial sources, such as Integrated DNA Technologies (Skokie, Ill.), Operon Biotechnologies, Inc. (Huntsville, Ala.), and Invitrogen Corporation (Carlsbad, Calif.).

Preferred polymers have a length of at least 10 nucleosides, linked together through phosphodiester bonds. More preferably, polymers may have a length in the range of 20 to 100 nucleosides. Longer polymers are also possible, where two or more polymers are ligated together using an appropriate ligase enzyme (for example, DNA ligase, RNA ligase, among others). Longer polymers may also be prepared by rolling cycle polymerization (Mao et al. 2005). However generated, polymers of the present invention may include any number of reactive substituents that are incorporated typically as synthetic nucleotidyl units (for example, modified phosphoramidites) during synthesis of the polymer structure. The polymers may also include reactive groups located at the 5′ and/or 3′ termini, which may be incorporated either during synthesis of the polymer or added to the termini post-synthesis using an appropriate enzyme (for example, polynucleotide kinase, RNA ligase, terminal deoxynucleotidyl transferase, poly(A) polymerase, among others). Such reactive groups may include reactive substituents suitable for reacting with another reagent (for example, a linking reagent) or another species capable of directly coupling to a nanomaterial; for ligating two polymers together; or for coupling the polymer to a surface (for example, a surface fixing reagent).

The surface of a nanomaterial also may be modified to permit its attachment to the assembly template. Surfaces of nanomaterials may be chemically modified using a variety of functional groups for reaction with the linker agent of the assembly template. Examples of such modifications include thiols, amines, carboxylic acids, and aldehydes. The surface of a gold particle, for example, can be modified with a sulfhydryl-containing agent containing a nucleophile (for example, a protected form of 2-aminoethanethiol, such as 2-maleimidoethanethiol) that can react with the linking agent of the assembly template (for example, succinimidyl iodoacetate). Any protecting group present on the surface of the nanoparticle can be removed following completion of the surface modification reaction. Similarly, the surface of quantum dots, such as CdS and ZnS, can be modified to contain functional groups, such as amino or carboxyl groups for reaction with assembly templates. Additional examples of surface modifications suitable for nanomaterials are described in U.S. patent application Ser. No. 10/463,833, entitled SURFACE MODIFIED PROTEIN MICROPARTICLES, to Suslick et al., filed Jun. 17, 2003. In this regard, both the nanomaterial and the linking agent can be suitably modified to tailor the design of multifunctional nanoassemblies with exacting specificity.

The assembly template includes a polymer template and a linker agent. The linker agent may display a coupling activity preference for a surface group functionality of a nanomaterial. The coupling activity preference may be determined as ratio of the binding specificities that the reactive group of the linker agent displays for two surface group functionalities. For example, a thiol reactive group of a linker agent may display a greater binding specificity for a gold-coated surface relative to a SiO₂⁻ coated surface. A coupling activity preference for an assembly template containing an available thiol linker agent for these two coated surfaces may be determined in the following manner. The assembly template may be immobilized onto a resin matrix through one of the polymer termini, and resins lacking or containing the assembly template are incubated with solution mixtures containing equimolar amounts of gold and silver metals. The free and bound fractions of the metals are recovered by separating the solution from the resins. The amount of each metal that is present in each fraction can be determined by procedures known in the art, such as elemental analysis using atomic absorption spectroscopy or electron microscopy. After correction for the amount of each metal that binds non-specifically to a control resin lacking the assembly template, the coupling activity preference of the reactive group of the linker agent for gold relative to silver would then be calculated as the ratio of percentages of the respective metal ligands that bind specifically to the assembly template.

Preferably, the linker agent will display a coupling activity preference for a nanomaterial having a specific surface group functionality (for example, a gold coating) that is greater than 2-fold, such as in the range of 5-fold to 100-fold, relative to nanomaterial having a different surface group functionality (for example, a silver coating). More preferably, the linker agent will display a coupling activity preference greater than 10-fold, such as in the range of 20-fold to 80-fold. Most preferably, the linker agent will display a coupling activity preference greater than 25-fold, such as in the range of 40-fold to more than 100-fold.

Multifunctional nanoassemblies may be manufactured in single-pot syntheses in a variety of ways. Preferably, assembly templates may be prepared that contain linker agents having markedly different reactive group specificities for different nanomaterial surfaces. Where assembly templates contain two different types of linker agents having respective coupling activity preferences of 10-fold for their respective nanomaterials, the coupling of the nanomaterials to the appropriate linker agents with selectivity more than 100-fold is possible. Where assembly templates contain different types of linker agents having comparable or no coupling activity preference for their respective nanomaterials, the use of linker agents having different protecting groups may permit sequential assembly of nanomaterials onto the assembly template following sequential deprotection of the linker agents. In this manner, separate classes of nanomaterials may be coupled to specific linker agents based upon whether the reactive group of the linker agent is deprotected and available for the coupling reaction.

Single-pot syntheses of multifunctional nanoassemblies may be performed in solution or on solid-phase supports. Preferably, assembly templates are coupled to solid phase supports prior to reaction with nanomaterials. The use of solid-phase support media offers several important advantages over conventional solution chemistries, including improved reaction efficiencies, washing procedures to remove free non-coupled nanoparticles, and recovery of the desired nanoassemblies. Where single-pot syntheses require sequential deprotection of individual classes of linker agents before nanomaterials are coupled, the use of solid-phase support medium offers the additional advantage of including capping reactions to exclude uncoupled linker agents of the prior coupling reaction cycle from participating in unwanted coupling reactions during subsequent cycles of nanomaterial addition to the assembly template.

In a manner analogous to cycling methods used for automated nucleic acid synthesis on solid-phase supports, automated programs may be designed for multifunctional nanoassembly synthesis that employ individual cycles that include discrete steps, such as a linker agent deprotection step, a first wash step, a nanomaterial addition step, a nanomaterial coupling reaction step, an unreacted nanomaterial removal step, a second wash step, a capping reagent addition step, a capping reaction step, an unreacted capping reagent removal step, and a third wash step. The automated programs may include options for specifying time and temperature conditions for individual steps of each cycle as well. As a final step of the automated synthesis program, the multifunctional nanoassembly may be released from the solid-phase support column and subjected to further purification as needed.

Long, one-dimensional, polymers may be formed by self-assembly using a single strand (Mao et al. 2006). Two-dimensional and three-dimensional DNA structures may also be formed by self-assembly, and these are the preferred structures for nanoassemblies (Seeman 2003; Chen et al. 1991; Endo et al. 2005; Winfree et al. 1998; Yang et al. 1998; Chelyapov et al. 2004; Yan et al. 2003; Lund et al. 2005; and Goodman et al. 2005).

Where formation of nanomaterials is driven by a template-directed self-assembly process, such as by hybridization of partially or fully complementary polymers, multifunctional assemblies may be achieved using assembly templates that contain only one modification on the polymer. Since two-dimensional polymer nanostructures may be composed of a plurality of polymer strands (for example, thirty or more strands), each of the assembly templates may be separately formed where each polymer contains a reactive substituent at a particular location that is coupled to a different linker using different linking reagents. For example, assembly template A may contain a BIBDE linker at position 15, assembly template B may contain a biotin linker at position 35, assembly template C may contain an aptamer linker at position 62, and assembly template D may contain an antibody linker at position 80. These assembly templates may be combined together to form a larger assembly, provided that sufficient complementarity exists among the assembly templates or that suitable complementary polymers are provided in the mixture containing the assembly templates. Once assembled, the larger assembly may be reacted with different nanomaterials, each of which may contain an appropriate coating suitable for coupling to the specific linker. For example, with regard to the aforementioned assembly templates A-D, a Au nanomaterial may be coupled to the thiol moiety of the BIBDE linker of assembly template A; a nanomaterial containing avidin may be coupled to the biotin linker of assembly template B; a nanomaterial containing an aptamer ligand may be coupled to the aptamer linker of assembly template C; and a nanomaterial containing an antigen may be coupled to the antibody linker of assembly template D. In this example, the multifunctional nanoassembly may be formed in a one-pot synthesis owing to the unique specificities of the nanomaterial-linker coupling reactions. The nanomaterials also may be coupled to the individual assembly templates before the multifunctional assemblies are formed.

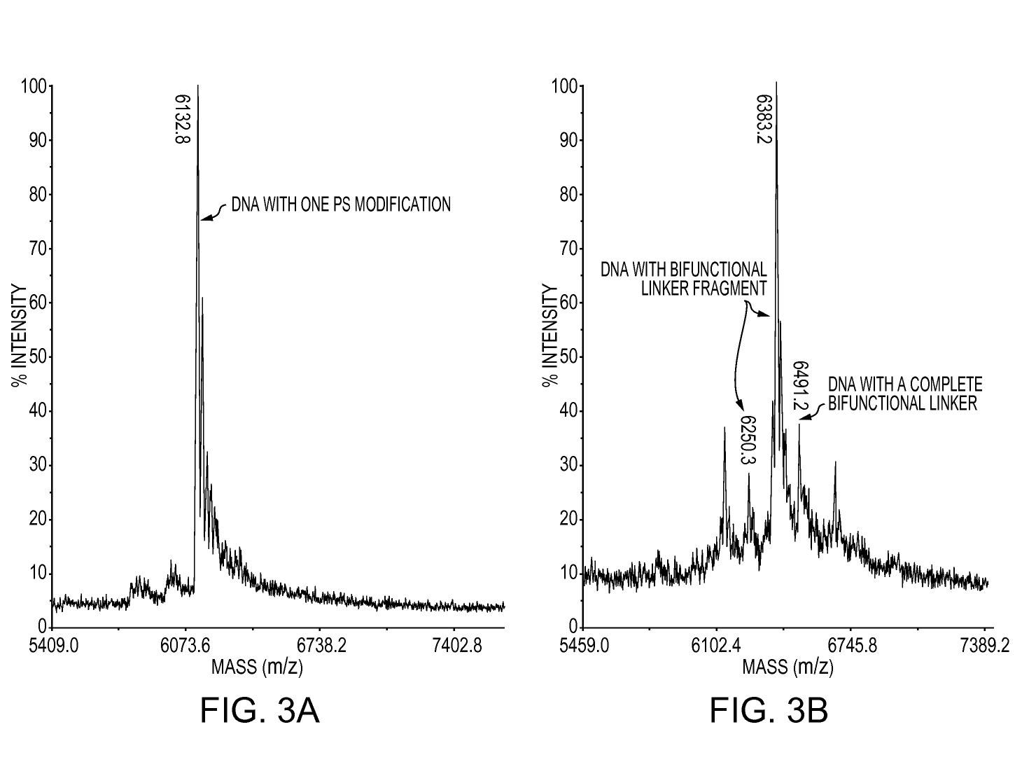

One can use a variety of methods to monitor the manufacture of nanoassemblies and multifunctional nanoassemblies. The inclusion of the reactive substituent at one or more defined positions in the polymer may be monitored by mass spectrometry, as the apparent molecular mass of the polymer parent ion will change due to incorporation of the reactive substituent. FIG. 3(a) depicts an example of the mass of a polymer containing a phosphorothioate as a reactive substituent. For certain reactive substituents, such as phosphorothioate, the location of the reactive substituent may be confirmed by modification and cleavage of the polymer by iodine and sizing the resultant polymer cleavage products according to any mass size detection method known in the art (for example, size exclusion chromatography, PAGE, mass spectrometry, among others). The coupling of the linking reagent to the polymer can be monitored by mass spectrometry, as the apparent molecular mass of the polymer parent ion will increase due to the presence of the linking agent (FIG. 3(b)). The ability of nanoassemblies to form regular secondary and tertiary structures may be studied by thermal denaturation analysis using UV-vis spectroscopy, by imaging using atomic force microscopy, and/or circular dichroism, among others. The coupling of the nanomaterial to the assembly template may be analyzed by scanning electron microscopy, by mobility shift assay, as well as by other methods commonly known in the art.

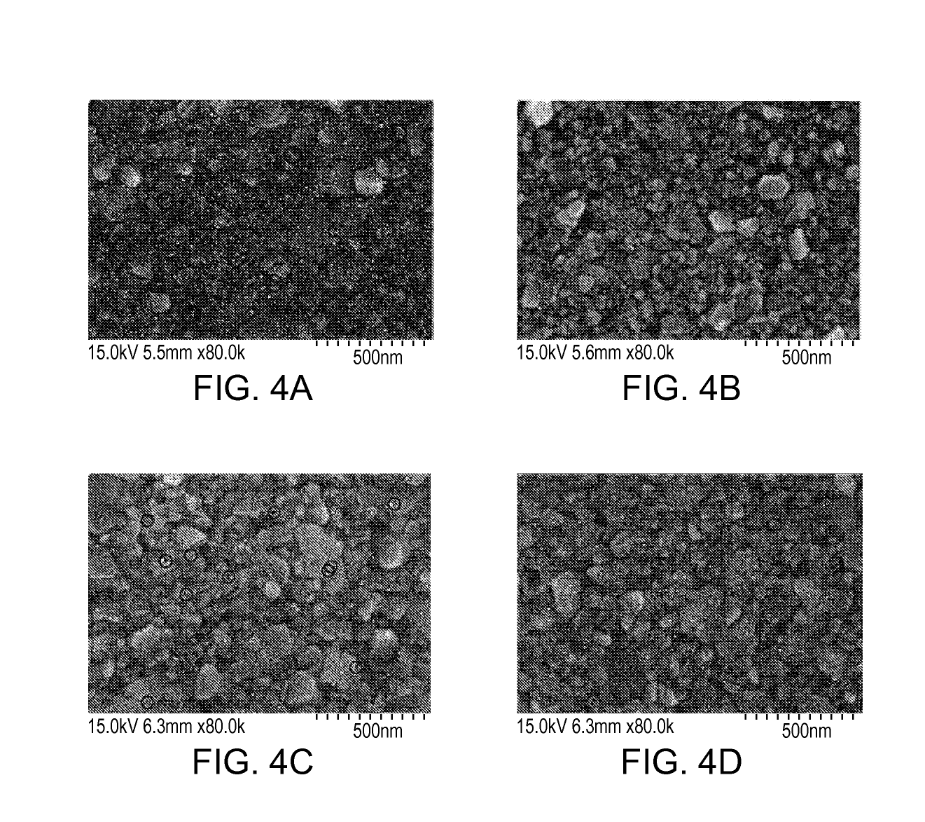

The nanoassemblies may be fixed onto a variety of surfaces as the intended application warrants. Preferably, the nanoassemblies are bound onto a two-dimensional surface. The nanoassemblies may be attached to the surface using chemical modifications to the polymer structure, such as through linkage of a surface fixing reagent to the 5′ and/or 3′ terminus of the polymer of the nanoassembly. For example, a nanoassembly containing a double-stranded polymer that has its 5′-termini modified to contain a thiol permits immobilization of the nanoassembly onto Au thin film on silicon wafers. Scanning electron microscopic analysis can be used to confirm the attachment of the nanoassembly to the surface and to characterize the structure of the nanoassembly (FIG. 4). DNA can also be immobilized on freshly cleaved mica surface by divalent metal ions, such as Mg²⁺, Ni²⁺, and Zn²⁺, or on aminopropylsilane modified mica surface, and be imaged by Atomic Force Microscopy (AFM) (Liu, Z. et al. 2005).

The nanomaterials can be attached to an assembly template before or after the assembly template is attached to a target surface. In cases where the assembly template is attached to the target surface prior to attachment of the nanomaterial, the linking agent of the assembly template should remain protected to prevent the reactive group of the linking agent from chemically reacting with the target surface. Following attachment of the assembly template to the target surface, the linking agent of the assembly template is deprotected to permit reaction with the desired nanomaterial.

While the foregoing disclosure is specifically directed the methods that permit precise coupling of a nanomaterial on a modified phosphodiester linkage at a defined position in a nucleic acid polymer and the generation of multifunctional nanoassemblies, the methods are also generally applicable to micromaterials, such as microparticles (for example, polystyrene, silica, and titania microparticles). Thus, the methods of the present invention contemplate assemblies that include nanomaterials, micromaterials, and mixtures thereof.

### EXAMPLES

### Example 1

### Assembly Template Synthesis

The polymers used herein can be prepared according to standard nucleic acid synthesis procedures. The specific polymers listed in Table 1 were purchased from Integrated DNA Technologies and subjected to gel purification.

TABLE 1
Nucleic acid polymers¹

| | |
|---|---|
| SEQ ID NO: 1 | 5′-TT*T* TTA GCA TAT GAC TAT GTT ACT CGC TAT AGC-3′ |
| SEQ ID NO: 2 | 5′-GTA CTT GCA ATA TGT GCA ATG GCG AGG ATT T*T*T-3′ |
| SEQ ID NO: 3 | 5′-AAT CCT CGC CAT TGC ACA TAT TGC AAG TAC GCT ATA |
| | GCG AGT AAC ATA GTC ATA TGC TAA-3′ |
| SEQ ID NO: 4 | 5′-AAT CGT ATA CTG ATA CAA TGA GCG ATA TCG CAT GAA |
| | CGT TAT ACA CGT TAC CGC TCC TAA-3′ |
| SEQ ID NO: 5 | 5′-CG*G CAT GCA* T-3′ |
| SEQ ID NO: 6 | 5′-AT*G CAT GCC* G-3′ |
| SEQ ID NO: 7 | 5′-GTG CAG ACT* T-3′ |
| SEQ ID NO: 8 | 5′-AAG TCT GCA C-3′ |
| SEQ ID NO: 9 | 5′-GTG CAG A*CC TTG TGA ACG CC-3′ |
| SEQ ID NO: 10 | 5′-GGC GTT C*AC AAG GTC TGC AC-3′ |

¹An asterisk indicates the location of a reactive substituent (phosphorothioate) in the designated polymer.

Assembly templates were prepared with linking reagents BIDBE and monobromobimane.

(a) Assembly Templates Prepared with BIDBE

The linking reagent BIDBE was prepared using the method of Luduena et al. (1981). Briefly, 16.2 mg of cystamine dihydrochloride was dissolved in 4 mL of 0.1 N NaOH; 77.8 mg of iodoacetic anhydride was dissolved in 1 mL of 1,2-dichloroethane; and the two solutions were combined and agitated on a vortex mixer for 1 minute to form a white precipitate. The pellets were collected by centrifugation and dried under vacuum for 1 hour. The pellets were dissolved in acetone and centrifuged to remove any precipitated iodoacetate by-product. The supernatant was collected and dried under stream of argon gas to obtain BIDBE as a white powder.

The coupling of BIDBE to the polymer to form the assembly template and the reduction of the disulfide bond of BIDBE following its conjugation to a polymer was accomplished with TCEP in the following manner. The BIDBE linkers can be coupled to phosphorothioate modified DNA polymers either before or after DNA hybridization to form double-stranded DNA. The choice of method depends on reaction conditions. Since the linker labeled on phosphorothioate modified DNA is not stable enough at high pH or at high temperature, care should be taken to avoid extreme conditions during or after linker modification.

When low pH is acceptable and DNA can be easily hybridized in a short period of time, for example, in a simple case of double stranded DNA formation, linker can be coupled to the single stranded DNA first and then hybridized to its complementary DNA at lower pH (pH 5-6 or lower). The procedures are as follows: to 36 μL of 10 mM phosphate buffer (pH 7), 10 μL of 1 mM phosphorothioate-modified DNA (SEQ ID NO: 7) in water was added, together with 20 μL of 100 mM BIDBE solution in DMF. The pH of the solution does not affect the reaction yield, as long as the pH is kept in the range between pH 5 to 8. The reaction yield depends heavily on the ratio between linker and the number of phosphorothioate modification on DNA. If the phosphorothioate modification on the DNA increases from 1 to n, the concentration of the DNA should be lowered by n-fold accordingly. The optimum ratio between linker and DNA is ˜200. The reaction was carried out at 50° C. for ˜5 hours, after which, the excess amounts of linkers and DMF in the solution is removed via a gel filtration column (PD-10 column).

To hybridize DNA to form linker-containing double stranded DNA template, 10 μL of 500 nM single-stranded linker-labeled DNA prepared above was mixed with 10 μL of 500 nM complementary DNA (SEQ ID NO: 8) in 20 mM acetate (or citrate) buffer (pH 5) containing 50 mM NaCl and 2 mM EDTA. Before annealing, 4 μL of 100 mM TCEP was added to the above mixture and the solution was let to stand at room temperature for 15-60 minutes to allow TCEP to reduce the disulfide bond in the BIDBE linker or to cleave possible cross-linked DNA molecules formed due to the symmetric structure of the BIDBE linker. Annealing was then carried out by heating the above mixture to ˜95° C. and cooling down the solution to room temperature in ˜2 hours.

The addition of TCEP is important in this case because the single-stranded DNA folded by BIDBE linkers is impossible to be hybridized with complementary DNA unless the linkers are cleaved and single-stranded linker-labeled DNA becomes unfolded by adding TCEP. The TCEP was not intentionally removed after reaction with linker since it is better to keep the solution under reducing conditions for subsequent reactions with nanoparticles.

To form more complex 2D or 3D DNA structures, higher pH (pH 7-8) and longer annealing process (˜48 hours) are necessary. Under these conditions, it is preferable to first form double-stranded DNA and then couple the linker to the DNA template structures. The procedures are as follows: DNA solution containing 100 μM of both complementary DNAs (SEQ ID NOS: 9 and 10) in 10 mM acetate buffer (pH 5) with 50 mM NaCl and 2 mM EDTA were annealed for desirable hours to ensure complete hybridization. The pH of the solution does not affect the reaction yield, as long as the pH is kept in the range between pH 5 to 8. After annealing, 100 mM BIDBE solution in DMF was added so that the final solution contains about 30% (vol/vol) BIDBE solution. This gave the optimum ratio between linkers and phosphorothioate modifications in both DNAs (about 200:1). The double-stranded DNA was reacted with the linker at 50° C. for ˜5 hours and then slowly cooled down to make sure DNA stay hybridized. The TCEP is not necessary when linker is coupled to double-stranded DNA as the DNA already exists in a hybridized form. The reaction yield was greater than 90%. After the reaction, the excessive linker and DMF could be removed by running through gel filtration column (PD-10 column). The DNA remained double-stranded after column purification. Even though this latter method is similar to the former method of coupling the linker to single-stranded DNA first before forming double-stranded DNA, this latter method gives much better yield, especially under harsher conditions, resulting in much less linker removal from DNA, loss of phosphorothioate group (that is, conversion of sulfur to oxygen), or internal DNA cleavage. Thereafter, the template was reacted with TCEP reduce the disulfide bond in the BIDBE linker and cleave other products as described above.

When preparing the 2D or 3D DNA network, it may be unnecessary to remove excessive linkers and DMF using gel filtration columns. The formation of DNA network and imaging via AFM were not substantially affected by the presence of DMF or excess BIDBE linker very much.

(b) Assembly Templates Prepared with Monobromobimane

The reaction between phosphorothioate DNA (SEQ ID NOS: 5 and 6) and monobromobimane was made by modification of the procedure of Fidanza et al. (1992). Briefly, monobromobimane was dissolved in DMF to prepare a 13.3 mM monobromobimane solution. This solution (62.9 ml) was mixed with 32.3 μL of 1 mM phosphorothioate DNA and 114 μL of 10 mM phosphate buffer (pH 7). Following reaction at 25° C. for 1 hour (70-75% yield), the assembly template was purified on a PD-10 column to remove the unreacted monobromobimane. A control experiment confirmed that the DNA lacking phosphorothioate modification has no reactivity with monobromobimane.

### Example 2

### Conjugation of a Nanoparticle to an Assembly Template

Unconjugated 5 nm Au nanoparticles were purchased from TED PELLA (Redding, Calif.) and were stabilized by conjugating the Au nanoparticle surface with phosphine ligand. Typically, 20 mL of 83 nM Au nanoparticles were stirred with 4 mg bis(para-sulfonatophenyl)phenylphosphine dihydrate dipotassium salt (Strem Chemicals, Newburyport, Mass.) and kept shaking at room temperature for longer than 10 hours. To remove the excessive salt in the solution, Au nanoparticles were precipitated with sufficient amount of NaCl until the color changed from red to blue and the supernatant were removed after centrifugation. The Au nanoparticles were redispersed in deionized water (MILLIPORE™) to a concentration of 500 nM. A 150-200 nM Au nanoparticle solution was prepared in 10 mM acetate buffer (pH 5) containing 10 mM NaCl and 2 mM TCEP.

The TCEP is necessary to reduce the BIDBE linker before assembling Au nanoparticles on DNA because the thiol in the bifunctional linker will not be exposed unless the disulfide bond is reduced. Since TCEP was already used when labeling BIDBE linker on single-stranded DNA during the DNA hybridization process, smaller amounts of TCEP may be used (for example, 1 mM) but it is preferred to maintain the solution under the reducing environment for the reaction of the DNA with nanoparticles.

A thin film surface (50 nM-thick deposited Au by thermal evaporator on Si wafer with 5 nm Cr thin film in between as a buffer layer) which had phosphorothioate modified DNA immobilized on it was incubated in the Au nanoparticle solution for 2-3 hours. After nanoassembly containing Au nanoparticle conjugated to the DNA assembly plate was made, the Au surface was washed thoroughly with 10 mM NaCl, 10 mM acetate buffer (pH 5).

### Example 3

### Characterization of a Nanoassembly by UV-Vis Spectroscopy

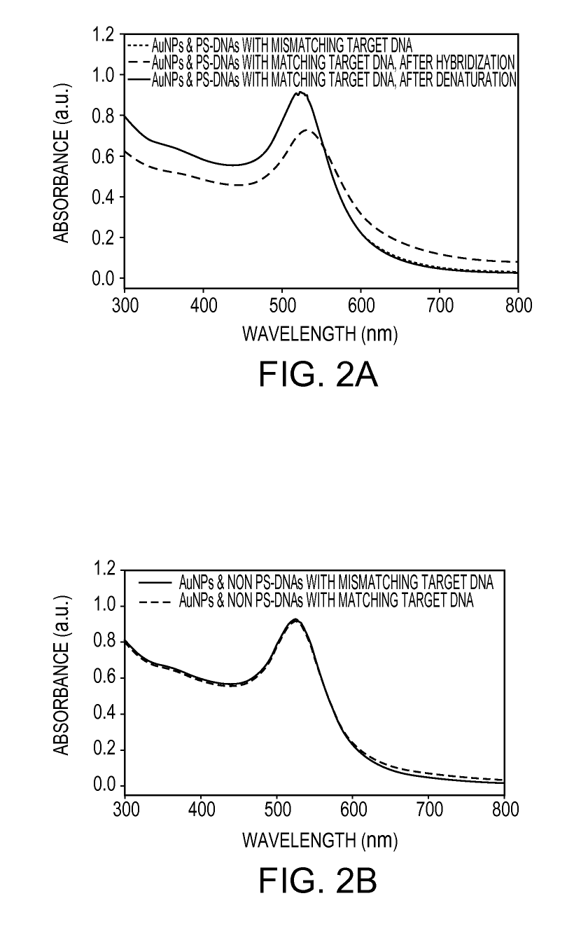

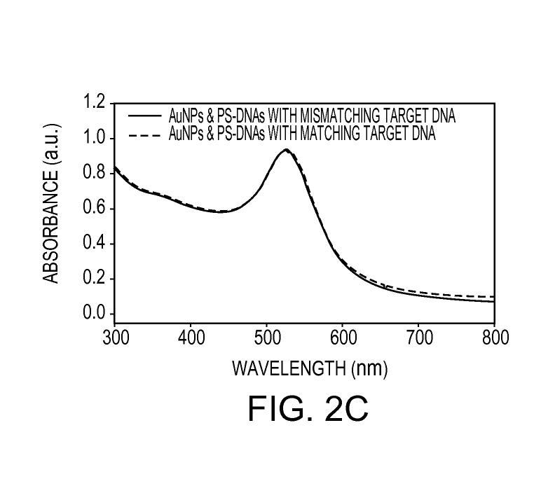

An aggregation and disassembly experiment was conducted with nanoparticle assemblies to illustrate that the Au nanoparticles were conjugated to the assembly template. Thirteen nm Au nanoparticles were synthesized by the citrate reduction method. The Au nanoparticles were functionalized by an assembly template comprising a 33-nucleotide oligonucleotide that included two adjacent end position phosphorothioate modifications (SEQ ID NO. 1), each of which underwent reaction with the linking reagent, BIDBE. A second analogously modified but non-complementary 33-nucleotide oligonucleotide (SEQ ID NO. 2) was used to functionalize another population of 13 nm Au nanoparticles. The functionalized Au nanoparticles remained dispersed when mixed but then aggregated in the presence of a bridging target DNA strand (SEQ ID NO. 3), which is complementary to both assembly template strands (see FIG. 2A). The Au nanoparticles disassembled when heated above the 33-mer melting temperature, and these Au nanoparticle aggregation/disassembly behaviors were repeatable. By contrast, the Au nanoparticle assembly was not observed when a non-bridging mismatch DNA strand (SEQ ID NO. 4) was used. These results demonstrate that the assembly template is bound to Au nanoparticles and that the Au nanoparticle assembly behaviors can be controlled by the polymer in a fashion similar to that observed for alkane thiol modified DNA. Gold nanoparticles mixed with DNA polymer lacking a reactive substituent, such as phosphorothioate, did not show aggregation properties (FIG. 2B). Likewise, Au nanoparticles mixed with a polymer lacking chemical reaction with the linking reagent did not show aggregation properties (FIG. 2C). These experiments confirm that the presence of at least one reactive substituent in the polymer and its reaction with a linking reagent are necessary in order to successfully attach Au nanoparticles to an assembly template.

### Example 4

### Characterization of a Nanoassembly by Scanning Electron Microscopy

The following example illustrates the precise control of Au nanoparticle positioning using assembly templates containing phosphorothioate incorporated at specific sites and reacted with the linking reagent BIDBE. Five nanometer Au nanoparticles were assembled on assembly templates that were immobilized onto a 50 nm thick Au thin film on silicon wafer and micrographs were collected by SEM. In order to increase the yield of Au NPs binding to phosphorothioate modified sites and to make the linkage rigid, three adjacent phosphate moieties were modified to phosphorothioate to bind with a single Au NP. To form a Au nanoparticle trimer with a 40 base pair gap between nanoparticles, 100-mer DNA was used with position 9, 10, 11, 49, 50, 51, 89, 90, and 91 nucleotides modified with phosphorothioates that had been reacted with BIDBE. After purifying the DNA polymer using a PD-10 column, the single-stranded polymer was hybridized to a complementary DNA strand containing an alkane thiol modification on the 5′ end with sufficient reducing reagent TCEP to reduce the disulfide bond of BIDBE. The alkane thiol group on the complementary DNA serves as a surface fixing reagent to attach the double-stranded polymer to Au thin films not only to image with SEM but also to remove any Au nanoparticles that are not coupled to the assembly template. Control experiments showed that most of double-stranded polymer was immobilized on the surface via the alkane thiol modification on the end of the complementary DNA rather than those extended from bifunctional linkers on phosphorothioate modifications. Phosphorothioate modifications were positioned in the middle of the assembly template strand so that alkane thiol groups on the bifunctional linkers have a reduced chance to bind to the Au film compared to those at the end of the double-stranded polymer. The phosphorothioate modifications were designed to face up after DNA immobilization on the wafer surface. After 3 hours of incubation in the 5 nm Au nanoparticle solution, the Au nanoparticles were attached to phosphorothioate-modified polymer by the linker segment.

The SEM images show that the surface-immobilized double-stranded polymer containing three triplet phosphorothioate modifications at 40 base-pair intervals [3PS-DNA(40, 40)] has a large amount of Au NPs bound on the surface (FIG. 4a). In contrast, the control surface-immobilized with double-stranded polymer that lacks the reactive substituent phosphorothioate [0PS-DNA] has little to no Au nanoparticles. The occurrence of any Au nanoparticles in this image is likely attributed to nonspecific binding of Au nanoparticles to the Au surface (FIG. 4b). Having shown that the phosphorothioate modifications in [3PS-DNA(40, 40)] are necessary for Au nanoparticle positioning, the Au NPs should be present only at those triplet phosphorothioate sites. Therefore, trimers of Au NPs should retain the spacing of 40 base-pairs (−13.6 nm) when assembled by [3PS-DNA(40, 40)]. Furthermore, dimers with 70 base-pairs (24 nm) and 50 base-pairs (17 nm) distances between Au nanoparticles, which are assembled on a DNA polymer containing two triplet phosphorothioate modifications with 70 base-pair interval [2PSDNA(70)] and 50 base-pair interval [2PS-DNA(50)], respectively, should enable determination of position specificity. Indeed, the predicted controlled spacings of 13.6 nm, 24 nm, and 17 nm were achieved, as shown in FIG. 4a, c, and d, respectively. As the length of the double-stranded polymer (−34 nm) used for assembly is below the persistence length (−50 nm), the distances between Au nanoparticles can be directly compared to the distances between phosphorothioate modifications of the polymer.

### REFERENCES

1. Aldaye, F. A. et al., “Sequential self-assembly of a DNA hexagon as a template for the organization of gold nanoparticles” Angew Chem Int Ed Engl., 45, pp. 2204-09, Mar. 27, 2006.
2. Alivisatos, A. P. et al.; “Organization of ‘nanocrystal molecules’ using DNA”, Nature, 382, pp. 609-11, Aug. 15, 1996.
3. Alivisatos, A. P. et al, “DNA-Based Assembly of Gold Nanocrystals”, Angewandte Chemie, International Edition, 38, pp. 1808-12, Jun. 14, 1999.
4. Carbone, A. et al., “Circuits and programmable Self-Assembling DNA Structures”, Proceedings of the National Academy of Sciences of the United States of America, 99, pp. 12577-82, Oct. 1, 2002, Epub Sep. 13, 2002.
5. Chelyapov, N. et al., “DNA triangles and self-assembled hexagonal tilings”, J. Am. Chem. Soc., 126, pp. 3924-25, Nov. 3, 2004.
6. Chelyapov, N. et al., “DNA Triangles and Self-Assembled Hexagonal Tilings”, J. Am. Chem. Soc., 126, pp. 13924-25, Nov. 3, 2004.
7. Chen, J. H. et al., “Synthesis from DNA of a molecule with the connectivity of a cube”, Nature, 350, pp. 631-33, Apr. 18, 1991.
8. Conway, N. E. et al., “The Covalent Attachment Of Multiple Fluorophores To DNA Containing Phosphorothioate Diesters Results In Highly Sensitive Detection Of Single-Stranded DNA”, Bioconjugate Chem, 2, pp. 452-57, November-December 1991.
9. Deng, Z. et al., “DNA-encoded self-assembly of gold nanoparticles into one-dimensional arrays”, Angew Chem Int Ed Engl., 44, pp. 3582-85, Jun. 6, 2005.
10. Ding, B. et al., “Pseudohexagonal 2D DNA Crystals from Double Crossover Cohesion”, J. Am. Chem. Soc., 126, pp. 10230-31, Aug. 25, 2004.
11. Endo, M. et al., “DNA tube structures controlled by a four-way-branched DNA connector”, Angew Chem Int Ed Engl., 44, pp. 6074-77, Sep. 19, 2005.
12. Fidanza J. A. et al. “Site-specific labeling of DNA sequences containing phosphorothioate diesters”, J. Am. Chem. Soc., 114, 5509-5517, 1992.
13. Goodman, R. P. et al., “Rapid chiral assembly of rigid DNA building blocks for molecular nanofabrication”, Science, 310, pp. 1661-65, Dec. 9, 2005.
14. Hagleitner, C. et al., “Smart single-chip gas sensor microsystem”, Nature, 414, pp. 293-96, Nov. 15, 2001.
15. He, Y et al., “Sequence Symmetry as a Tool for Designing DNA Nanostructures”, Angew Chem Int Ed Engl, 44, pp. 6694-96, Oct. 21, 2005.
16. Heath, J. R. et al., “A Defect-Tolerant Computer Architecture: Opportunities for Nanotechnology”, Science, 280, pp. 1716-19, Jun. 12, 1998.
17. Holloway, G. et al., “An organometallic route to oligonucleotides containing phosphoroselenoate”, Chem Bio Chem 3, pp. 1061-65, Nov. 4, 2002.
18. Li, H. et al., “DNA-templated self-assembly of protein and nanoparticle linear arrays”, J. Am. Chem. Soc., 126, pp. 418-419, Jan. 21, 2004.
19. Lu, Y. et al., “Spectroscopic Evidence for Inner-Sphere Coordination of Metal Ions to the Active Site of a Hammerhead Ribozyme”, J. Am. Chem. Soc., 120, pp. 4518-19, Apr. 28, 1998.
20. Luduena, R. F., et al., N,N-Bis(□-iodoacetyl)-2,2′-dithiobis(ethylamine), a reversible crosslinking reagent for protein sulfhydryl groups. Anal Biochem., 117(1):76-80, October, 1981.
21. Lund, K. et al., “Self-assembling a molecular pegboard”, J. Am. Chem. Soc., 127, pp. 17606-607, Dec. 21, 2005.
22. Lund, K., et al., “Self-Assembling a Molecular Pegboard”, J. Am. Chem. Soc., 127, 17606-07, Dec. 21, 2005.
23. Mathieu, F. et al. “Six-Helix Bundles Designed from DNA”, Nano Lett., 5, 661-65, Apr. 5, 2005.
24. Mao, C. et al., “DNA-Encoded Self-Assembly of Gold Nanoparticles into One-Dimensional Arrays”, Angewandte Chemie, International Edition, 44, pp. 3582-85, Jun. 6, 2005.
25. Mao, C. et al, “Approaching The Limit: Can One DNA Oligonucleotide Assemble into Large Nanostructures?”, Angewandte Chemie, 118, pp. 1976-79, Mar. 13, 2006.
26. McLaughlin, L. W. et al., “Introduction of reporter groups at specific sites in DNA containing phosphorothioate Jesters”, J. Am. Chem. Soc., 111, pp. 9117-19, Dec. 6, 1989.
27. McLaughlin, L. W. et al., “Site-Specific Labeling of DNA Sequences Containing Phosphorothioate Diesters” J. Am. Chem. Soc., 114, pp. 5509-17, Jul. 1, 1992.
28. Nakao, H. et al, “Highly Ordered Assemblies of Au Nanoparticles Organized on DNA”, Nano. Letters, 3, pp. 1391-94, Sep. 20, 2003.
29. Patolsky, F. et al., “Au-nanoparticle nanowires based on DNA and polylysine templates”, Angew Chem Int Ed Engl., 41, pp. 2323-27, Jul. 2, 2002.
30. Pinto, Y. et al., “Sequence-Encoded Self-Assembly of Multiple-Nanocomponent Arrays by 2D DNA Scaffolding” Nano Lett., 5, pp. 2399-402, Dec. 5, 2005.
31. Rothemund, P., “Folding DNA to create nanoscale shapes and patterns”, Nature, 440, pp. 297-302, Mar. 16, 2006.
32. Seeman, N. C. et al, “Ligation of DNA Triangles Containing Double Crossover Molecules”, J. Am. Chem. Soc., 120, pp. 9779-86, Sep. 9, 1998.
33. Seeman, N.C., “Nucleic Acid Nanostructures and Topology”, Angewandte Chemie, International Edition, 37, pp. 3220-38, Dec. 17, 1998.
34. Seeman, N.C., “At the Crossroads of Chemistry, Biology, and Materials: Structural DNA Nanotechnology”, Chemistry & Biology, 10, pp. 1151-59, Dec. 10, 2003.
35. Seeman, N. C., “DNA in a material world”, Nature, 421, pp. 427-31, Jan. 23, 2003.
36. Seeman, N.C., “DNA-Templated Self-Assembly of Metallic Nanocomponent Arrays on a Surface”, Nano Lett., 4, pp. 2343-47, Oct. 29, 2004.
37. Seeman, N. C. et al. “Nucleic acid nanostructures: bottom-up control of geometry on the nanoscale”, Rep. Prog. Phys., 68, pp. 237-270, 2005. (published Dec. 6, 2004).
38. Warner, M. G. et al.; “Linear assemblies of nanoparticles electrostatically organized on DNA scaffolds.” Nature Materials, 2, pp. 272-77, April 2003.
39. Winfree, E. et al., “Design and self-assembly of two-dimensional DNA crystals”, Nature, 394, pp. 539-44, Aug. 6, 1998.
40. Woehrle, G. H. et al.; “Molecular-level control of feature separation in one-dimensional nanostructure assemblies formed by biomolecular nanolithography”, Langmuir, 20, 5982-88, Jul. 6, 2004.
41. Yan, H. et al. “DNA-Templated Self-Assembly of Protein Arrays and Highly Conductive Nanowires”, Science, 301, pp. 1882-84, Sep. 26, 2003.
42. Yan, H. et al., “DNA-templated self-assembly of protein arrays and highly conductive nanowires”, Science, 301, pp. 1882-84, Sep. 26, 2003.
43. Yan, H. et al, “Periodic Square-Like Gold Nanoparticle Arrays Templated by Self-Assembled 2D DNA Nanogrids on a Surface”, Nano Lett., 6, 248-51, Jan. 17, 2006.
44. Yang, T. et al. “Tunneling Phase Logic Cellular Nonlinear Networks”, Int. J. Bifurc. Chaos, 11, pp. 2895-911, December, 2001.
45. Liu, Z. et al., “Imaging DNA Molecules on Mica Surface by Atomic Force Microscopy in Air and in Liquid”, Microsc. Res. Tech., 66, pp 179-185, March, 2005

1.-46. (canceled)

47. A method of generating an assembly with a desired linear, two-dimensional or three-dimensional structure, comprising:
(a) selecting one or more internal positions of a nucleic acid polymer template for attachment of a particle;
(b) predicting a linear, two-dimensional and three-dimensional structure of the nucleic acid polymer template when the particle is attached to the one or more selected internal positions of the nucleic acid polymer;
(c) reacting the nucleic acid polymer template with the particle,
wherein the nucleic acid polymer template comprises:
a single-stranded molecule or a double-stranded molecule comprising DNA, RNA, PNA, or mixed co-polymers thereof, and
one or more modified phosphodiester linkages at the selected one or more internal positions within the single stranded molecule or within one or both strands of the double-stranded molecule, wherein the one or more modified phosphodiester linkages each comprise a reactive substituent,
and wherein the particle comprises at least one linking reagent comprising a first reactive group and a second reactive group separated by a linker segment, wherein the second reactive group is attached the particle,
under conditions that permit the first reactive group to attach to the reactive substituent, and
(d) coupling the particle to the one or more selected internal positions of the nucleic acid polymer template through the linking reagent, thereby forming the assembly with the desired linear, two-dimensional or three-dimensional structure.

48. The method of claim 47, further comprising synthesizing the nucleic acid polymer template, wherein synthesizing comprises introducing the reactive substituents at the selected one or more internal positions.

49. The method of claim 47, wherein the reactive substituent comprises phosphorothioate, phosphoselenoate, or phosphoroamide.

50. The method of claim 47, wherein the linking reagent comprises dithio-bis-succinimidyl propionate (Lomant's reagent), N-succinimidyl-(4-iodoacetyl)aminobenzoate (SIAB), 3-maleimidopropionic acid (NHS), sulfo-SIAB, N-succinimidyl S-acetylthioacetate, N-succinimidyl S-acetylthiopropionate succinimidyl iodoacetate, succinimidyl bromoacetate, succinimidyl-6-(iodoacetyl)aminocaproate, N,N′-Bis (α-isoacetyl)-2,2′-dithiobis(ethylamine), bromo-α,β-unsaturated carbonyls, iodo (or bromo) acetamides, aziridinylsulfonamides, 3-(2-iodoacetamido)-2,2,5,5-tetramethyl-1-pyrrolidinyloxy (PROXYL), monobromobimane, 4-bromocrotonic acid, γ-bromo-α,β-unsaturated carbonyl dihydropyrroloindole, bromoacetamido dihydropyrroloindole, or N-dansylaziridin.

51. The method of claim 47, further comprising reacting the nucleic acid polymer template with a reducing agent prior to reacting the nucleic acid polymer template with the particle.

52. The method of claim 51, wherein the reducing agent comprises tris(2-carboxyethyl)phosphine hydrochloride.

53. The method of claim 47, further comprising reacting the nucleic acid polymer template with a surface fixing reagent to form a surface-reactive nucleic acid polymer template.

54. The method of claim 53, further comprising reacting the surface-reactive nucleic acid polymer template with a surface to attach the nucleic acid polymer template to the surface.

55. The method of claim 47, wherein the particle is a nanoparticle.

56. The method of claim 55, wherein the nanoparticle is a nanorod, nanosphere, nanotube, nanofiber, nanowire, nanobelt, nanosheet, nanocard, nanoprism, or quantum dot.

57. The method of claim 54, wherein the nanoparticle comprises a gold nanoparticle.

58. The method of claim 47, wherein the particle is a microparticle,

59. The method of claim 58, wherein the microparticle is a microrod, microsphere, microtube, microfiber, microwire, microbelt, microsheet, microcard, or microprism.

60. The method of claim 47, wherein the assembly is fixed onto a surface.

61. The method of claim 47, wherein:
the nucleic acid polymer template further comprises a second reactive substituent positioned at the 5′ and/or 3′ termini of the nucleic acid polymer template, and
the assembly further comprises a second linking reagent, wherein the second linking reagent is attached to the second reactive substituent.

62. The method of claim 47, wherein the method prepares a multifunctional assembly, and wherein reacting the nucleic acid polymer template with the particle comprises reacting the nucleic acid polymer template with a first particle and a second particle, wherein the first and second particles are different types of particles.

63. The method of claim 62, wherein the first particle and the second particle comprise a first linking reagent and a second linking reagent, respectively,
wherein the first linking reagent comprises a first reactive group and a second reactive group separated by a linker segment, wherein the first reactive group is attached to a first reactive substituent on the nucleic acid polymer template and the second reactive group is attached the first particle, thereby coupling the nucleic acid polymer template to the first particle through the linking reagent; and
wherein the second linking reagent comprises a third reactive group and a fourth reactive group separated by a linker segment, wherein the third reactive group is attached to a second reactive substituent on the nucleic acid polymer template and the fourth reactive group is attached the second particle, thereby coupling the p nucleic acid polymer template to the second particle through the linking reagent.

64. The method of claim 62, wherein the multifunctional assembly comprises a multifunctional nanoassembly, wherein the first and second particles are different types of nanoparticles.

65. The method of claim 62, wherein the multifunctional assembly comprises a multifunctional microassembly, wherein the first and second particles are different types of microparticles.

66. The method of claim 47, wherein the linker segment separates the first reactive group and the second reactive group by a distance from 2 Å to 50 Å.

67. The method of claim 47, wherein the linker segment comprises linear and branched alkyl groups, saturated and unsaturated alkyl groups, amides, amines, ethers, or esters.

68. The method of claim 47, wherein the nucleic acid polymer template is 20 to 100 nucleosides in length.

69. The method of claim 47, wherein the nucleic acid polymer template comprises reactive substituents at two selected internal positions of the nucleic acid polymer, and the predicted structure is a:
a lariat polymer structure where the two selected internal positions of the nucleic acid polymer are linked together through the linker segment;
a linear polymer structure that contains two linking agents singly-coupled at each selected internal position of the nucleic acid polymer; or
a multimeric polymer structure that contains two or more nucleic acid polymer templates crosslinked together through one or more linker segments.

70. An assembly made by the method of claim 47.

71. The method of claim 47, wherein the assembly comprises a plurality of nucleic acid polymer templates.
